(19) United States
(12) Patent Application Publication
(10) Pub. No.: US 2024/0401117 A1
(43) Pub. Date: Dec. 5, 2024

(54) METHODS FOR PREPARING HIGH-RESOLUTION SPATIAL ARRAYS

(71) Applicant: 10x Genomics, Inc., Pleasanton, CA (US)

(72) Inventor: Felice Alessio Bava, Rome (IT)

(21) Appl. No.: 18/804,654

(22) Filed: Aug. 14, 2024

Related U.S. Application Data
(63) Continuation of application No. 17/165,453, filed on Feb. 2, 2021, now Pat. No. 12,110,541.
(60) Provisional application No. 62/969,460, filed on Feb. 3, 2020.

(51) Int. Cl. C12Q 1/6837; C12Q 1/6874

(52) U.S. Cl. CPC C12Q 1/6837; C12Q 1/6874

(57) ABSTRACT

Provided herein are methods of preparing a spatial array and methods for associating specific sample analytes with spatial locations in the spatial array. Provided herein are methods for preparing a spatial array using a plurality of primers attached to a substrate to guide a plurality of features to specific locations on the spatial array. In a non-limiting example, a plurality of primers on a substrate can be used to guide a plurality of first features that include capture probes onto the substrate. A plurality of second features that are configured to hybridize to the first features and also include capture probes are added to the substrate.

### CROSS-REFERENCE TO RELATED APPLICATION

This application is a continuation to U.S. patent application Ser. No. 17/165,453, filed Feb. 2, 2021, which claims priority to U.S. Provisional Patent Application No. 62/969,460, filed Feb. 3, 2020. The contents of this application is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Spatial transcriptomics arrays can be designed a priori so that the position of each oligonucleotide (e.g., capture probe) is predetermined, with known X-Y positions for each oligonucleotide. However, resolution of printed spatial transcriptomics arrays can be increased. Bead-based arrays can reach higher resolution then printed arrays, but a decoding mechanism is needed to determine the position of each bead a posteriori. This is usually achieved through a decoding chemistry that requires the use of a dedicated instrument or system. Thus, there remains a need to develop arrays with increased resolution and without a decoding mechanism.

### SUMMARY

In one aspect, this disclosure includes methods for preparing a spatial array including: (a) providing a substrate including a plurality of primers attached to a surface of the substrate, where a primer of the plurality of primers includes a first hybridization domain; (b) providing a plurality of first features, where a feature of the plurality of first features includes an oligonucleotide, a first capture probe, and a first bridging probe; where: the oligonucleotide includes a second hybridization domain, where the second hybridization domain is capable of hybridizing to the first hybridization domain; the first capture probe includes a first spatial barcode and a first capture domain, where the first capture domain is capable of binding a first analyte; and the first bridging probe includes a first bridging domain, where the first bridging domain is capable of binding to a second bridging domain; attaching the plurality of first features to the plurality of primers by coupling the second hybridization domain to the first hybridization domain; and associating the first feature with a location in the spatial array based on the location of the hybridization domain of the primer. In some embodiments, the method further includes: (e) providing a plurality of second features, where a feature of the plurality of second features includes a second capture probe and a second bridging probe, where: the second capture probe includes a second spatial barcode and a second capture domain, where the second capture domain is capable of binding a second analyte; and the second bridging probe includes a second bridging domain, where the second bridging domain is capable of binding to the first bridging domain; (f) attaching the plurality of second features to the plurality of first features by coupling the second bridging probe to the first bridging probe; and (g) associating the first feature and the second feature with a location in the spatial array based on the location of the first spatial barcode and the second spatial barcodes in the spatial array.

In some embodiments, the primer is affixed to the substrate at a 5′ end of the primer.

In some embodiments, the primer is deposited onto the substrate in a manner where the primer has a known location (e.g., a predetermined deposition location) on the substrate. In some embodiments, the primers are deposited onto the substrate by printing (e.g., inkjet printing). In some embodiments, the primers are deposited onto the substrate by photolithography.

In some embodiments, the method further includes amplifying all or part of the primer. In some embodiments, the amplifying is isothermal. In some embodiments, the amplifying is not isothermal. In some embodiments, the isothermal amplification is rolling circle amplification. In some embodiments, the amplifying step is performed prior to step (b).

In some embodiments, the oligonucleotide further includes a cleavage domain. In some embodiments, the cleavage domain is a cleavable linker. In some embodiments, the cleavable linker is a photocleavable linker, a UV-cleavable linker, a chemically cleavable linker, or an enzymatic cleavable linker. In some embodiments, the cleavable linker is an enzymatic cleavable linker.

In some embodiments, the first bridging domain includes a sequence that is at least partially complementary to the second bridging domain. In some embodiments, the first bridging probe includes a first bridging domain having a sequence that is a different length compared to other bridging domains. In some embodiments, the second bridging probe includes a second bridging domain having a sequence that is a different length compared to other bridging domains.

In some embodiments, the method includes a step (e) that further includes increasing the spatial array temperature as compared to the spatial array temperature in steps (a)-(d), where the increase in temperature is associated with the sequence of the first bridging domain and the second bridging domain.

In some embodiments, the first bridging domain is about 10 nucleotides to about 30 nucleotides. In some embodiments, the first bridging domain is about 30 to about 50 nucleotides. In some embodiments, the first bridging domain is about 50 to about 70 nucleotides. In some embodiments, the first bridging domain is about 70 nucleotides to about 90 nucleotides. In some embodiments, the first bridging domain is at least 90 nucleotides.

In some embodiments, the second bridging domain is about 10 nucleotides to about 30 nucleotides. In some embodiments, the second bridging domain is about 30 to about 50 nucleotides. In some embodiments, the second bridging domain is about 50 to about 70 nucleotides. In some embodiments, the second bridging domain is about 70 nucleotides to about 90 nucleotides. In some embodiments, the second bridging domain is at least 90 nucleotides.

In some embodiments, the method further includes washing the substrate after step (d), thereby removing unattached first features from the spatial array. In some embodiments, the method further includes washing the substrate after step (f), thereby removing unattached second features from the spatial array.

In some embodiments, the method further includes providing a bridging domain blocking moiety that interacts with the first bridging domain or the second bridging domain. In some embodiments, the method further includes providing the bridging domain blocking moiety after step (c). In some embodiments, the method further includes releasing the bridging domain blocking moiety from the first bridging domain and/or second bridging domain prior to step (e).

In some embodiments, the first spatial barcode and the second spatial barcode are the same. In some embodiments, the first spatial barcode and the second spatial barcode are different. In some embodiments, the first capture domain and the second capture domain are the same. In some embodiments, the first capture domain and the second capture domain each include a poly(T) domain. In some embodiments, the first capture domain and the second capture domain are different.

In some embodiments, a feature of the plurality of first features includes a known combination of first capture probe, oligonucleotide, and first bridging probe, where determining the location of the first feature is based on the known combination.

In some embodiments, a feature of the plurality of second features includes a known combination of second capture probe and second bridging probe, where determining the location of the second feature is based on the known combination.

In some embodiments, the method further includes: (h) capturing a first analyte of a biological sample with a first capture probe of the plurality of first capture probes and/or a second capture probe of the plurality of second capture probes; and (i) determining a location of the first captured analyte in the biological sample based on the location of the first and/or second feature in the spatial array. In some embodiments, where capturing the first analyte of the biological sample with the first capture probe and/or the second capture probe includes contacting the spatial array with the biological sample and allowing the first analyte to interact with the first and/or second capture probe. In some embodiments, the determining step includes amplifying all or part of the first analyte specifically bound to the capture domain.

In some embodiments, the method further includes amplifying a portion of one of the plurality of first capture probes and/or second capture probes and/or analyte using isothermal amplification. In some embodiments, the method further includes amplifying a portion of one of the plurality of first capture probes and/or second capture probes and/or analytes using non-isothermal amplification. In some embodiments, the amplifying creates an amplification product including (i) all or part of a sequence of the analyte specifically bound to the first capture domain and/or the second capture domain, or a complement thereof, and (ii) all or part of the sequence of the first spatial barcode and/or the second spatial barcode, or a complement thereof.

In some embodiments, the associating step includes determining (i) all or part of the sequence of the first spatial barcode and (ii) all or part of the sequence of the second spatial barcode and using the determined sequence of (i) and (ii) to identify the location of the first feature and the location of the second feature in the spatial array.

In some embodiments, the determining step includes sequencing. In some embodiments, sequencing is performed via sequencing-by-synthesis (SBS), sequential fluorescence hybridization, sequencing by ligation (SBL), nucleic acid hybridization, or high-throughput digital nucleic acid sequencing techniques.

In some embodiments, the analyte is RNA or DNA.

In another aspect, this disclosure includes methods for spatial analysis of a biological analyte in a biological sample including: (a) preparing a spatial array by the method of any one of the methods described herein; (b) contacting the biological sample to the spatial array under conditions where the biological analyte binds a capture probe on the first feature and/or the second feature; (c) determining (i) all or a part of the sequence of the biological analyte specifically bound to the first capture domain and/or the second capture domain, or a complement thereof, and (ii) all or a part of the sequence of the first spatial barcode and/or the second spatial barcode, or a complement thereof, and using the determined sequence of (i) and (ii) to identify the location of the analyte in the biological sample.

In some embodiments, the method further includes extending the capture probes via a polymerization reaction using the biological analyte as a template to generate an extended capture probes including the capture probes and a reverse complement of the biological analyte.

In some embodiments, the feature of the plurality of first features is a first bead. In some embodiments, the feature of the plurality of second features is a second bead. In some embodiments, the first bead and/or the second bead has a diameter of about 0.1 μm to about 5 μm, about 1 μm to about 10 μm, about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 90 μm to about 100 μm, about 80 μm to about 100 μm, about 70 μm to about 100 μm, about 60 μm to about 100 μm, about 50 μm to about 100 μm, about 40 μm to about 100 μm, about 30 μm to about 100 μm, about 20 μm to about 100 μm, or about 10 μm to about 100 μm.

In another aspect, this disclosure includes compositions including a substrate that includes (a) a plurality of primers attached to a surface of the substrate, wherein a primer of the plurality of primers includes a first hybridization domain; and (b) a plurality of first features, wherein a feature of the plurality of first features includes an oligonucleotide, a first capture probe, and a first bridging probe, wherein: (i) the oligonucleotide includes a second hybridization domain, wherein the second hybridization domain is capable of hybridizing to the first hybridization domain; (ii) the first capture probe includes a first spatial barcode and a first capture domain, wherein the first capture domain is capable of binding to a first analyte from a biological sample; and (iii) the first bridging probe includes a first bridging domain, wherein the first bridging domain is capable of binding to a second bridging domain, wherein a feature of the first plurality of features is coupled to a primer of the plurality of primers via hybridization of the first hybridization domain to the second hybridization domain.

In another aspect, this disclosure includes compositions that includes (a) a plurality of primers attached to a surface of the substrate, wherein a primer of the plurality of primers includes a first hybridization domain; (b) a plurality of first features, wherein a feature of the plurality of first features includes an oligonucleotide, a first capture probe, and a first bridging probe, wherein: (i) the oligonucleotide includes a second hybridization domain, wherein the second hybridization domain is capable of hybridizing to the first hybridization domain; (ii) the first capture probe includes a first spatial barcode and a first capture domain, wherein the first capture domain is capable of binding to a first analyte from a biological sample; and (iii) the first bridging probe includes a first bridging domain, wherein the first bridging domain is capable of binding to a second bridging domain; and (c) a plurality of second features, wherein a feature of the plurality of second features includes a second capture probe and a second bridging probe, wherein: (i) the second capture probe includes a second spatial barcode and a second capture domain, wherein the second capture domain is capable of binding to a second analyte from the biological sample; and (ii) the second bridging probe includes a second bridging domain, wherein the second bridging domain is capable of binding to the first bridging domain, wherein a feature of the first plurality of features is coupled to a primer of the plurality of primers via hybridization of the first hybridization domain to the second hybridization domain, wherein a feature of the second plurality of features is coupled to a feature of the first plurality of features via hybridization of the second bridging domain to the first bridging domain.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, where the cleaved capture probe can enter a non-permeabilized cell and bind to target analytes within the cell.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C are schematics illustrating how streptavidin cell tags can be utilized in an array-based system to produce spatially-barcoded cells or cellular contents.

FIG. 7A is a schematic showing an exemplary feature hybridized to a primer (e.g., an oligonucleotide) on a substrate.

FIG. 7B is a schematic showing an exemplary second feature hybridized to a first feature.

### DETAILED DESCRIPTION

### I. Introduction

Spatial analysis methods using capture probes and/or analyte capture agents provide information regarding the abundance and location of an analyte (e.g., a nucleic acid or protein). Traditionally, these methods identify a singular molecule at a location. Extending these methods to study interactions between two or more analytes would provide information on the interactions between two or more analytes at a location in a biological sample. Analyte capture agents as provided herein comprises an analyte binding moiety affixed to an oligonucleotide. The oligonucleotide comprises a nucleic acid sequence that uniquely identifies the analyte and the analyte binding moiety. Further, nearby oligonucleotides affixed to a different analyte binding moiety in a nearby location can be hybridized to the first oligonucleotide and then can be detected using the spatial methods described herein. The methods disclosed herein thus provide the ability to study the interaction between two or more analytes at one or more locations in a biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2)ec0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, nuclei, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I) (d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

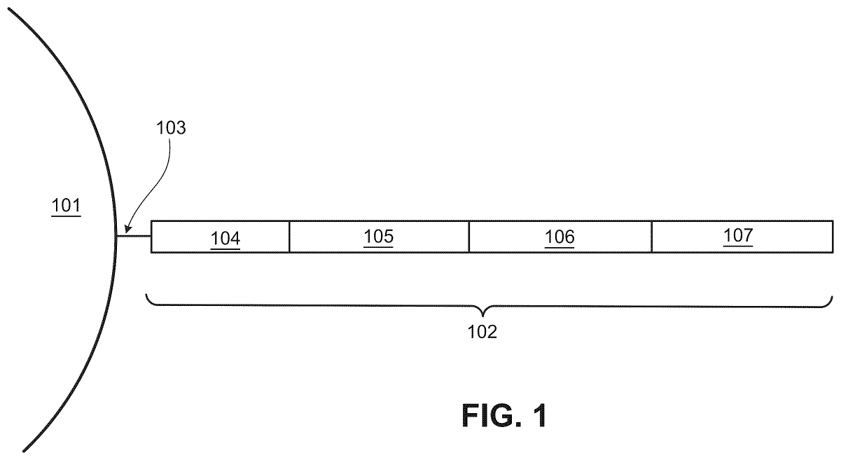

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

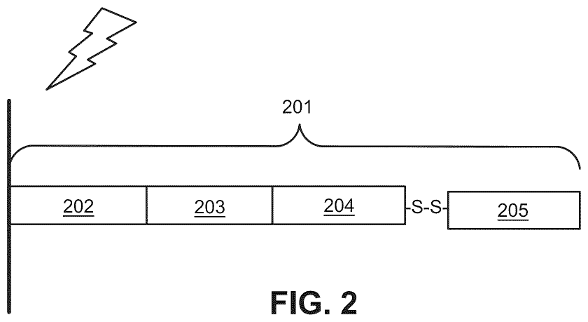

FIG. 2 is a schematic illustrating a cleavable capture probe, wherein the cleaved capture probe can enter a non-permeabilized cell and bind to analytes within the cell. The capture probe 201 contains a cleavage domain 202, a cell penetrating peptide 203, a reporter molecule 204, and a disulfide bond (—S—S—). 205 represents all other parts of a capture probe, for example a spatial barcode, a UMI and a capture domain.

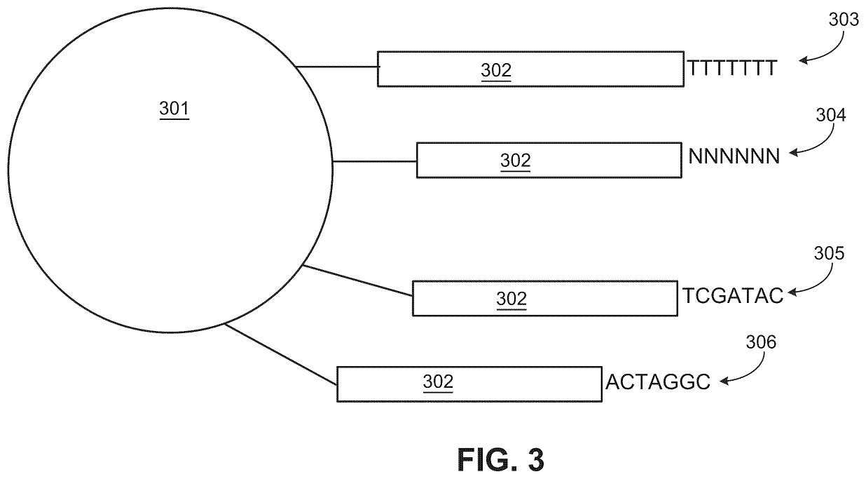

FIG. 3 is a schematic diagram of an exemplary multiplexed spatially-barcoded feature. In FIG. 3, the feature 301 can be coupled to spatially-barcoded capture probes, where the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302 and a different capture domain. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the scheme shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. Sec, e.g., Section (II) (b) (e.g., subsections (i)-(vi)) of WO 2020/176788 and/or U.S. Patent Application Publication No. 2020/0277663. Generation of capture probes can be achieved by any appropriate method, including those described in Section (II) (d) (ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

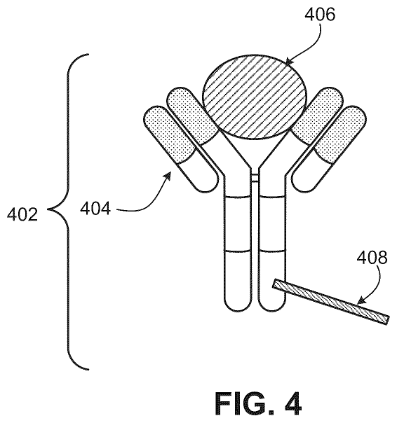

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

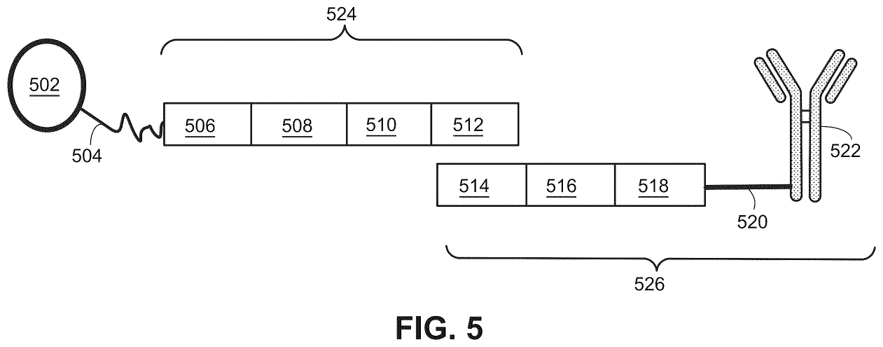

FIG. 5 is a schematic diagram depicting an exemplary interaction between a capture probe 524 immobilized on a feature 502 via a linker 504 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

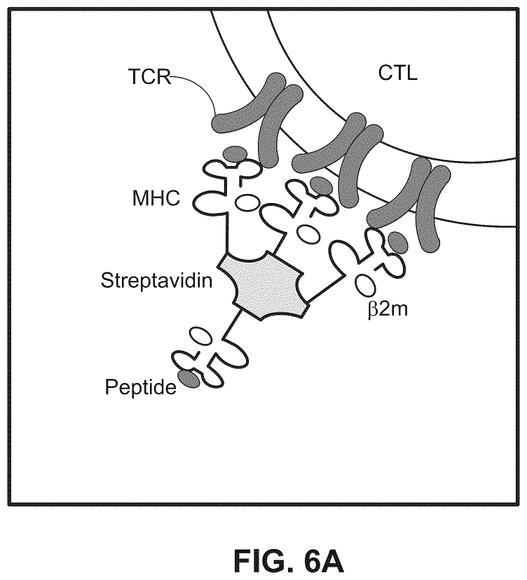

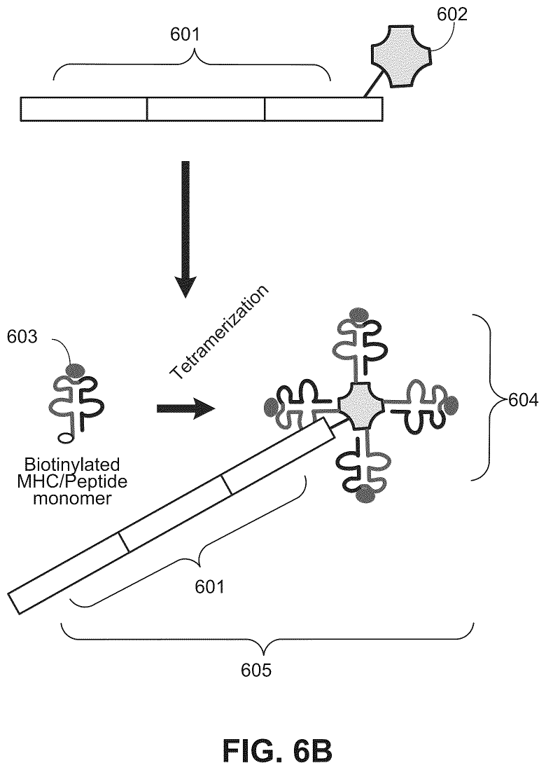

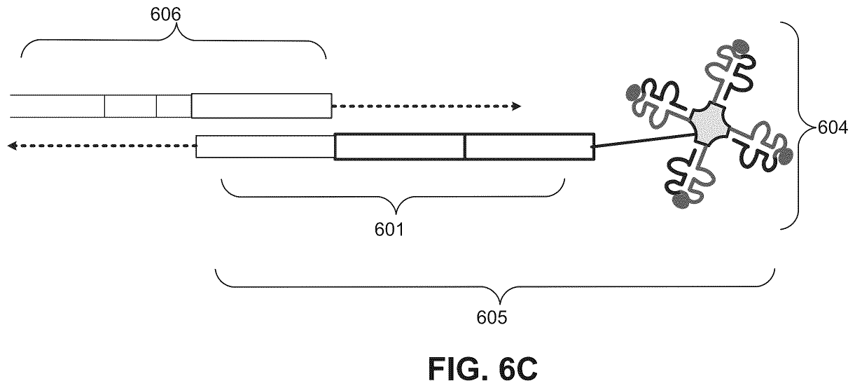

FIGS. 6A, 6B, and 6C are schematics illustrating how streptavidin cell tags can be utilized in an array-based system to produce a spatially-barcoded cell. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a T-Cell Receptor (TCR) such that the streptavidin binds to a target T-cell via multiple MCH/TCR binding interactions. Multiple interactions synergize and can substantially improve binding affinity. Such improved affinity can improve tagging of T-cells and also reduce the likelihood that tags will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated tag and also includes optional functional sequences such as sequences for hybridization with other oligonucleotides. As shown in FIG. 6C, one exemplary oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from the spatial barcode sequence on the capture agent barcode domain 601 may be used to identify the particular peptide MHC complex 604 bound to the cell (e.g., when using MHC-peptide libraries for screening immune cells or immune cell populations).

Additional description of analyte capture agents can be found in Section (II) (b) (ix) of WO 2020/176788 and/or Section (II) (b) (viii) U.S. Patent Application Publication No. 2020/0277663.

There are at least two methods to associate a spatial barcode with one or more neighboring cells (e.g., in a tissue sample), such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto cells of the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II) (a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of WO 2020/176788 and/or U.S. Patent Application Publication No. 2020/0277663. Some quality control measures are described in Section (II) (h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c) (ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Preparing a High-Resolution Spatial Array

### (a) Background

This disclosure includes methods for preparing a spatial array and methods for associating specific sample analytes with spatial locations in the spatial array. Provided herein are methods for preparing a spatial array using a plurality of primers attached to a substrate to guide a plurality of features to specific locations on the spatial array. In a non-limiting example, a plurality of primers on a substrate can be used to guide a plurality of first features that include capture probes onto the substrate to predetermined, or assigned, locations on the array. A plurality of second features that can hybridize to the first features and that also include capture probes can then be added to the substrate. The second set of features can hybridize to the first set of features. Thus, the position of the first set of features is determined by hybridization to the primers on the substrate, and the position of the second set of features is derived by their proximity to the first set of features. The second set of features can increase the resolution of the array as they can be deposited on the substrate at spaces between the primers and/or the first features. After any of the steps involving the first feature or the second feature, wash steps (e.g., using any of the methods described herein) can be used to remove unbound features. In some embodiments, the first and second features comprise beads. In some embodiments, the first and second features comprise same or different sizes, densities, masses, charges, etc. In some embodiments, the first and second features comprise beads of less than 25 microns average diameter. Also provided herein are methods that include using the spatial arrays to determine the location of an analyte in a biological sample.

The methods disclosed herein avoid the need of a decoding solution for random bead arrays; significantly simplify and provide and error-correction solution for bead-array decoding; and allow for amplification of a signal by transforming the signal of individual oligonucleotides (e.g., a single capture probe) into the signal of beads, where each bead is conjugated to a plurality (e.g., thousands or millions) of oligonucleotides (e.g., capture probes).

In some embodiments, a method for preparing a spatial array includes providing a substrate including a plurality of primers attached to a surface of the substrate, where a primer of the plurality of primers includes a first hybridization domain; contacting the substrate with a plurality of first features, where a feature of the plurality of first features includes an oligonucleotide that includes a second hybridization domain that includes a sequence that is substantially complementary to the first hybridization domain, a first capture probe that includes a first spatial barcode and a first capture domain, and a first bridging probe that includes a first bridging domain that includes a sequence that is substantially complementary to a second bridge domain; attaching the plurality of first features to the plurality of primers by coupling the second hybridization domain to the first hybridization domain; and associating the first feature with a location in the array based on the location of the hybridization domain of the primer.

In some embodiments, a method for preparing an array includes contacting the substrate with a plurality of second features, where the second features include a second capture probe that includes a second spatial barcode and a second capture domain and a second bridging probe that includes a second bridging domain that includes a sequence that is substantially complementary to the first bridging domain; attaching the plurality of second features to the plurality of first features by coupling the second bridging probe to the first bridging probe; and associating the first feature and the second feature with a location in the array based on the location of the first spatial barcode and the second spatial barcode on the array.

In some embodiments, a method for preparing a spatial array includes providing a substrate including a plurality of primers attached to a surface of the substrate, where a primer of the plurality of primers includes a first hybridization domain; contacting the substrate with a plurality of first features, where a feature of the plurality of first features includes an oligonucleotide that includes a second hybridization domain that includes a sequence that is substantially complementary to the first hybridization domain, a first capture probe that includes a first spatial barcode and a first capture domain, and a first bridging probe that includes a first bridging domain that includes a sequence that is substantially complementary to a second bridge domain; attaching the plurality of first features to the plurality of primers by coupling the second hybridization domain to the first hybridization domain; contacting the substrate with a plurality of second features, where the second features include a second capture probe that includes a second spatial barcode and a second capture domain and a second bridging probe that includes a second bridging domain that includes a sequence that is substantially complementary to the first bridging domain; attaching the plurality of second features to the plurality of first features by coupling the second bridging probe to the first bridging probe; and associating the first feature and the second feature with a location on the array based on the location of the first spatial barcode and the second spatial barcode on the array. In some embodiments, the associating step includes (i) associating the first feature with a location in the spatial array based on the location of the hybridization domain of the primer and (ii) associating the first feature and the second feature with a location in the spatial array based on the location of the first spatial barcode and the second spatial barcode on the array.

In some instances, the methods disclosed herein further include determining the abundance and location of the first analyte and/or the second analyte by the steps of contacting the spatial array with the biological sample; hybridizing the first analyte to the first capture probe and/or the second analyte to the second capture probe; and determining (i) all or a part of the sequence of the first analyte and/or the second analyte, or a complement thereof, and (ii) all or a part of the sequence of the first spatial barcode and/or the second spatial barcode, or a complement thereof, and using the determined sequence of (i) and (ii) to determine the abundance and the location of the first analyte and/or the second analyte in the biological sample.

### (b) Primer(s) on the Substrate

As used herein, a “primer” can refer to an oligonucleotide that is attached (e.g., affixed) to a substrate and includes a first hybridization domain that is capable of binding to a second hybridization domain. In some embodiments, a primer includes one or more sequences that are substantially complementary to a sequence on an oligonucleotide attached to a feature. In some cases, “primer” refers to the full length primer that is attached to the surface of the substrate and/or one or more constituent parts that make up a full length primer (e.g., a pool of nucleotides that will be synthesized together to make the full length primer and/or two or more sequences of nucleotides that can be ligated together to form the full length primer). As used herein, “full length primer” refers to a primer including at least a hybridization domain that is capable of binding to a second hybridization domain. As used herein a “primer array” can refer to a substrate that includes a plurality of primers attached (e.g., affixed) to the surface. In some embodiments, a primer array includes two or more sub-pluralities of primers. In such cases, each sub-plurality includes a different hybridization domain, a blocking probe attached to the hybridization domain, or both.

In some embodiments, the primer is about 10 to about 150 nucleotides (e.g., about 10, about 20, about 30, about 40, about 50, about 60, about 70, about 80, about 90, about 100, about 110, about 120, about 130, about 140, or about 150 nucleotides) in length. In some instances, the primer is a DNA molecule comprising DNA nucleotides (e.g., adenine (A), thymine (T), guanine (G), and cytosine (C)).

In some embodiments, a primer attached to a surface of a substrate is used to position one or more features on the substrate. In some embodiments, the primer includes a first hybridization domain. In some embodiments, the first hybridization domain includes a sequence at least partially complementary to the second hybridization domain. In some embodiments, the first hybridization domain includes a sequence that is substantially complementary to the second hybridization domain. In some embodiments, the first hybridization domain is at least 70% identical (e.g., at least 75% identical, at least 80% identical, at least 85% identical, at least 90% identical, at least 95% identical, or at least 99% identical) to the complementary sequence of the second hybridization domain.

In some embodiments, the primer is attached to the substrate in an orientation (e.g., attached via the 5′ end) such that the hybridization domain is on the free end (e.g., the free 3′ end). In some embodiments, a primer attached to a surface of a substrate also includes a functional sequence (e.g., any of the functional sequences described herein). For example, the functional sequence can be a sequence that binds an amplification primer, where the amplification primer can be used to amplify the primer attached to the surface of the substrate. In another example, the functional sequence can be a cleavage domain (e.g., any of the exemplary cleavage domains described herein). The cleavage domain can include a cleavable linker where a cleavable linker can include, without limitation, a photocleavable linker, a UV cleavable linker, a chemically cleavable linker or an enzymatic cleavable linker. In some embodiments, a primer attached to a surface includes a nucleic acid sequence or a nucleic acid tethered to henazine 5,10-di-N-oxide (see, e.g., Nagai and Hecht, J. Biol. Chem., 266 (35): 23994-4002 (1991), which is incorporated by reference in its entirety). When an antisense oligonucleotide anneals to a primer (e.g., a primer attached to a substrate) that includes a nucleic acid tethered to henazine 5,10-di-N-oxide, the primer can be contacted with a reducing agent (e.g., DTT), which generates oxygen radicals and effects strand scission of the primer, thereby resulting in cleavage of the primer.

In some embodiments, the primer includes from 5′ to 3′: a functional sequence (e.g., any of the exemplary functional sequences described herein) and a first hybridization domain. In some embodiments, the primer includes from 5′ to 3′: a first hybridization domain.

In some embodiments, the primer is affixed to the substrate via the 3′ end of the primer. In some embodiments, the primer includes from 3′ to 5′ a functional sequence (e.g., any of the exemplary functional sequences described herein) and a first hybridization domain. In some embodiments, the primer includes from 3′ to 5′ a first hybridization domain and a functional sequence (e.g., any of the exemplary functional sequences described herein). In some embodiments, the primer includes from 3′ to 5′ a first hybridization domain.

In some embodiments, a first hybridization domain is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 45 nucleotides, about 30 nucleotides to about 40 nucleotides, about 30 nucleotides to about 35 nucleotides, about 35 nucleotides to about 45 nucleotides, about 35 nucleotides to about 40 nucleotides, or about 40 nucleotides to about 45 nucleotides). In some embodiments, the length of the first hybridization domain can be used, at least in part, to deposit a feature on the substrate at a known location. In some embodiments, the sequence (i.e., the composition of nucleotides (A, G, C, and T)) of the primer can be used, at least in part, to deposit a feature on the substrate at a known location.

In some embodiments, a primer includes an affinity group. An “affinity group” is a molecule or molecular moiety which has a high affinity or preference for associating or binding with another specific or particular molecule or moiety. The association or binding with another specific or particular molecule or moiety can be via a non-covalent interaction, such as hydrogen bonding, ionic forces, and van der Waals interactions. An affinity group can, for example, be biotin, which has a high affinity or preference to associate or bind to the protein avidin or streptavidin. An affinity group, for example, can also refer to avidin or streptavidin which has an affinity to biotin. Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands. Any pair of affinity group and its specific or particular molecule or moiety to which it binds or associates with can have their roles reversed, for example, such that between a first molecule and a second molecule, in a first instance the first molecule is characterized as an affinity group for the second molecule, and in a second instance the second molecule is characterized as an affinity group for the first molecule.

In some embodiments, a primer includes an affinity group and an oligonucleotide on a feature of the first plurality of features includes a molecule for which the affinity group on the primer has a high affinity or preference for associating or binding. For example, without limitation, the primer can include a biotin affinity group and the oligonucleotide on the feature of the first plurality of features can include an avidin or streptavidin affinity group. In such cases, the biotin-avidin/streptavidin interaction hybridizes the feature of the first plurality of features to the primer attached to the substrate.

In some embodiments, a primer is deposited onto the substrate in a manner where the primer has a known or predetermined location on the substrate. In some embodiments, a primer is deposited onto the substrate at a known location on the substrate using synthesis (e.g., in situ synthesis), printing or lithography techniques.

In some embodiments, the primer is deposited on the substrate by “printing” or “spotting” (e.g., any of the exemplary printing methods described herein or known in the art (e.g., inkjet printing)). In some embodiments, the primer can be applied by either noncontact or contact printing. A noncontact printer can use the same method as computer printers (e.g., bubble jet or inkjet) to expel small (e.g., microliter, nanoliter or picoliter sized) droplets of primer solution onto the substrate. The specialized inkjet-like printer can expel nanoliter to picoliter volume droplets of primer solution onto the substrate. In contact printing, each print pin directly applies the primer solution onto a specific location on the surface. The primer can be attached to the substrate surface by electrostatic interaction of negatively charged phosphate backbone of DNA with a positively charged coating of the substrate surface or by UV-cross-linked covalent bonds between thymidine bases in the DNA and amine groups on the treated substrate surface. In some embodiments, the substrate is a glass slide. In some embodiments, the substrate is a semiconductor wafer (e.g., silicone wafer). In some embodiments, the primers are attached to a substrate by covalent attachment to a chemical matrix, e.g., epoxy-silane, amino-silane, lysine, polyacrylamide, etc.

In some embodiments, the primer is deposited on the substrate by photolithography. For example, light-directed synthesis of high-density DNA oligonucleotides can be achieved by photolithography or solid-phase DNA synthesis. In some embodiments, to implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical Microbiology Reviews 22.4 (2009): 611-633; US201314111482A; U.S. Pat. No. 9,593,365B2; US2019203275; and WO2018091676, which are each incorporated herein by reference in its entirety.

In some embodiments, primers can be prepared by in situ synthesis. In some embodiments, primer arrays can be prepared using photolithography-mediated synthesis. Photolithography typically relies on UV masking and light-directed combinatorial chemical synthesis on a substrate to selectively synthesize primers directly on the surface of an array, one nucleotide at a time per spot, for many spots simultaneously. In some embodiments, a substrate contains covalent linker molecules that have a photo-protecting group on the free end that can be removed by light. UV light can be directed through a photolithographic mask to deprotect and activate selected sites with hydroxyl groups that initiate coupling with incoming protected nucleotides that attach to the activated sites. The mask can be designed such that exposure sites can be selected, and thus specify the coordinates on the array where each nucleotide can be attached. The process can be repeated, and optionally a new mask is applied activating different sites and coupling different bases, allowing different oligonucleotides to be constructed at each site. This process can be used to synthesize hundreds of thousands of different primers (oligonucleotides). In some embodiments, maskless array synthesizer technology can be used to create an array. For example, programmable micromirrors can create digital masks that reflect a desired pattern of UV light to deprotect sites on a substrate similar to the mask method described above.

In some embodiments, inkjet spotting processes can be used for in situ oligonucleotide synthesis. Different nucleotide precursors plus a catalyst can be printed on the substrate, and are then combined with coupling and deprotection steps to create primers. This method relies on printing picoliter volumes of nucleotides on the array surface in repeated rounds of base-by-base printing that extends the length of the oligonucleotide primers on the array.

Primer arrays can also be prepared by active hybridization via electric fields to control nucleic acid (i.e., full length primers or the constituent parts of a full length primer) transport. Negatively charged nucleic acids can be transported to specific sites, or features, when a positive current is applied to one or more test sites on the array. The surface of the primer array can contain a binding molecule, e.g., streptavidin, which allows for the formation of bonds (e.g., streptavidin-biotin bonds) once electrically addressed biotinylated primers reach their targeted location. The positive current can then be removed from the active features, and new test sites can be activated by the targeted application of a positive current. The process can be repeated until all sites on the array are completed.

In some embodiments, a primer array can be generated through ligation of a plurality of oligonucleotides (e.g., the constituent parts of a full-length primer). In some instances, an oligonucleotide of the plurality contains a portion of a hybridization domain, and the complete hybridization domain is generated upon ligation of the plurality of oligonucleotides (e.g., each oligonucleotide includes a constituent part of a full-length primer). For example, a primer containing a first portion of a hybridization domain can be attached to a substrate (e.g., using any of the methods of attaching an oligonucleotide to a substrate described herein), and a second primer containing a second portion of the hybridization domain can then be ligated onto the first oligonucleotide to generate a complete hybridization domain. Different combinations of the first, second and any additional portions of a hybridization domain can be used to increase the diversity of the hybridization domains.

Primers can be generated by directly ligating additional oligonucleotides onto existing oligonucleotides via a splint oligonucleotide. In some embodiments, primers on an existing array can include a recognition sequence that can hybridize with a splint oligonucleotide. The recognition sequence can be at the free 5′ end or the free 3′ end of an oligonucleotide on the existing array. Recognition sequences useful for the methods of the present disclosure may not contain restriction enzyme recognition sites or secondary structures (e.g., hairpins), and may include high contents of Guanine and Cytosine nucleotides. When using a splint oligonucleotide to assist in the ligation of additional oligonucleotides, an additional oligonucleotide can include a sequence that is complementary to the sequence of the splint oligonucleotide. Ligation of the oligonucleotides to create a full-length primer can involve the use of an enzyme, such as, but not limited to, a ligase. Non-limiting examples of suitable ligases include Tth DNA ligase, Taq DNA ligase, Thermococcus sp. (strain 9oN) DNA ligase (9oN™ DNA ligase, New England Biolabs), Ampligase™ (available from Lucigen, Middleton, WI), and SplintR (available from New England Biolabs, Ipswich, MA). An array generated as described above is useful for spatial analysis of a biological sample. For example, one or more capture domains on the array can hybridize to poly(A) tails of mRNA molecules. Reverse transcription can be carried out using a reverse transcriptase to generate cDNA complementary to the captured mRNA. The sequence and location of the captured mRNA can then be determined (e.g., by sequencing the capture probe that contains the spatial barcode as well as the complementary cDNA).

Primers can also be generated by adding single nucleotides to existing oligonucleotides on an array, for example, using polymerases that function in a template-independent manner. Single nucleotides can be added to existing oligonucleotides in a concentration gradient, thereby generating primers with varying length, depending on the location of the primers on the array.

Primer arrays can also be prepared by modifying existing arrays, for example, by modifying oligonucleotides already attached to an array. For instance, primers (e.g., primers including a hybridization domain) can be generated on an array that already comprises oligonucleotides that are attached to the array (or features on the array) at the 3′ end and have a free 5′ end. In some instances, an array is any commercially available array (e.g., any of the arrays available commercially as described herein). The primers can be in situ synthesized using any of the in situ synthesis methods described herein.

An array for spatial analysis can be generated by various methods as described herein. In some embodiments, the array has a plurality of primers comprising hybridization domains that can hybridize to features that includes capture probes, where the capture probes include spatial barcodes and capture domains. These spatial barcodes and their relationship to the locations on the array can be determined.

In some embodiments, the primer attached to the surface of the substrate is functionalized. For example, the primer can include one or more functional groups. In such cases, the functional group can be used to control and shape the binding behavior and/or orientation of the primer, e.g., the functional group can be placed at the 5′ or 3′ end of the primer or within the sequence of the primer. Non-limiting examples of functional groups include amine-functionalized nucleic acids.

In some embodiments, the method of producing a spatial array further includes amplifying all or part of the primer. In some embodiments, amplification of all or part of the primer occurs prior to, contemporaneously with, or after the first set of features are provided to the spatial array. In some embodiments, the amplifying is isothermal. In some embodiments, the isothermal amplification is rolling circle amplification. In some embodiments, the amplifying is not isothermal. In some embodiments, the functional sequence includes a sequence capable of binding to a primer used for amplification (referred to herein as the “amplification primer” or “primer used for amplification”). In some embodiments, the amplification primer is used to amplify all or part of the primer attached to the substrate. In some embodiments, the amplification primer can be used to initiate a rolling circle amplification reaction. In some embodiments where a primer attached to the surface of the substrate is amplified, the amplification is performed by rolling circle amplification. In some embodiments, the primer to be amplified includes sequences (e.g., functional sequences, and/or hybridization sequences) that enable rolling circle amplification. In some embodiments, the substrate is contacted with an oligonucleotide (e.g., a padlock probe). As used herein, a “padlock probe” can refer to an oligonucleotide that has, at its 5′ and 3′ ends, sequences that are complementary to adjacent or nearby target sequences on a primer. Upon hybridization to the primer, the two ends of the padlock probe are either brought into contact or an end is extended until the two ends are brought into contact, allowing circularization of the padlock probe by ligation (e.g., ligation using any of the methods described herein (e.g., using a T4 DNA ligase)). In some embodiments, after circularization of the oligonucleotide, rolling circle amplification can be used to amplify the primer, which includes at least a hybridization domain from the primer. In some embodiments, amplification of the primer using a padlock oligonucleotide and rolling circle amplification increases the number of hybridization domains on the substrate.

In some embodiments, the effect of the amplification of all or part of the primer is to increase the number of first hybridization domains. For example, amplification of all or part of the primer using rolling circle amplification increases the number of first hybridization domains. The increased number of first hybridization domains in turn increases the number of sites to which the first features can couple to the primers thereby increasing the number of first features that can attach to the spatial array.

In some embodiments, the plurality of primers includes sub-pluralities that have different lengths of first hybridization domains. For example, a first sub-plurality (e.g., comprising about 50% of the total of the plurality of primers) includes a hybridization domain having a length of about 30 nucleotides and a second sub-plurality (e.g., comprising about 50% of the total of the plurality of primers) includes a hybridization domain having a length of about 70 nucleotides. In such cases, the first sub-plurality having a hybridization domain with a length of about 30 nucleotides can have a lower annealing temperature than the second sub-plurality having a hybridization domain with a length of about 70 nucleotides. The difference in annealing temperature can be used to encourage hybridization of a feature of a plurality of features to the first sub-plurality of primers over the second sub-plurality of primers, or vice versa.

In some embodiments, the first hybridization domain includes a sequence that is a different length compared to other hybridization domains. In some embodiments, the second hybridization domain includes a sequence that is a different length compared to other hybridization domains. In some embodiments, the first hybridization domain and the second hybridization domain are both about 10 nucleotides to about 30 nucleotides in length. In some embodiments, the first hybridization domain and the second hybridization domain are both about 30 nucleotides to about 50 nucleotides in length. In some embodiments, the first hybridization domain and the second hybridization domain are both about 50 nucleotides to about 70 nucleotides in length. In some embodiments, the first hybridization domain and the second hybridization domain are both about 70 nucleotides to about 90 nucleotides in length. In some embodiments, the first hybridization domain and the second hybridization domain are both at least 90 nucleotides in length.

In some embodiments, the method of producing the spatial array includes temperature modulation to encourage or discourage coupling of the first hybridization domain to the second hybridization domain (e.g., temperature modulation based on nucleotide sequence length). In some embodiments, annealing temperature is used to modulate the coupling of the first hybridization domain to the second hybridization domain. In some embodiments, the difference in annealing temperature can be used to encourage hybridization between a first hybridization domain and a second hybridization domain that have similar annealing temperatures. In some embodiments, a first hybridization domain and a second hybridization domain each have an annealing temperature that is about 35° C. to about 45° C., about 36° C. to about 44° C., about 37° C. to about 43° C., about 38° C. to about 42° C., or about 39° C. to about 41° C. In some embodiments, a first hybridization domain and a second hybridization domain each have an annealing temperature that is about 45° C. to about 55° C., about 46° C. to about 54° C., about 47° C. to about 53° C., about 48° C. to about 52° C., or about 49° C. to about 51° C. In some embodiments, a first hybridization domain and a second hybridization domain each have an annealing temperature that is about 55° C. to about 65° C., about 56° C. to about 64° C., about 57° C. to about 63° C., about 58° C. to about 62° C., or about 59° C. to about 61° C.

In some embodiments, the method includes providing a first hybridization domain blocking moiety. In some embodiments, the first hybridization domain blocking moiety prevents the first hybridization domain from binding (e.g., coupling) to the second hybridization domain either by binding to the first hybridization domain, second hybridization domain, or both. In some embodiments, the first hybridization domain blocking moiety needs to be removed before the first hybridization domain and second hybridization domain can be coupled. Non-limiting examples of methods to remove the first hybridization domain blocking moiety from binding to the first hybridization domain, second hybridization domain, or both include denaturation (e.g., increase in temperature), chemical (e.g., DTT) or enzymatic cleavage (e.g., nuclease). In some embodiments, the first hybridization domain blocking moiety is removed through passive means. For example, the binding affinity of the first hybridization domain is higher for the second hybridization domain than it is for the first hybridization domain blocking moiety. In such cases, the second hybridization domain out competes the first hybridization domain blocking moiety for binding to the first hybridization domain.

In some embodiments, the first hybridization domain blocking moiety is at least partially complementary to the first hybridization domain. In some embodiments, the first hybridization domain blocking moiety is at least at least 70% identical (e.g., at least 75% identical, at least 80% identical, at least 85% identical, at least 90% identical, at least 95% identical, or at least 99% identical) to the complementary sequence of the first hybridization domain. In some embodiments, binding of the first hybridization domain blocking moiety to the first hybridization domain blocks the coupling of the first hybridization domain to the second hybridization domain. In some embodiments, the method includes releasing (e.g., releasing using any of the methods described herein or know in the art) the first hybridization domain blocking moiety from the first hybridization domain.

In some embodiments, the method includes providing a second hybridization domain blocking moiety. In some embodiments, the second hybridization domain blocking moiety prevents the second hybridization domain from binding (e.g., coupling) to the first hybridization domain either by binding to the second hybridization domain. In some embodiments, the second hybridization domain blocking moiety is at least partially complementary to the second hybridization domain. In some embodiments, the second hybridization domain blocking moiety is at least at least 70% identical (e.g., at least 75% identical, at least 80% identical, at least 85% identical, at least 90% identical, at least 95% identical, or at least 99% identical) to the complementary sequence of the second hybridization domain. In some embodiments, binding of the second hybridization domain blocking moiety to the second hybridization domain blocks the coupling of the second hybridization domain to the first hybridization domain. In some embodiments, the method includes releasing (e.g., releasing using any of the methods described herein or know in the art) the first hybridization domain blocking moiety from the second hybridization domain.

### (c) Oligonucleotide(s) on a Feature of the Plurality of First Features

In some embodiments, a feature of the plurality of first features includes an oligonucleotide (or a plurality of oligonucleotides) that includes a second hybridization domain. In some embodiments, a feature of plurality of first features includes an oligonucleotide that includes a second hybridization domain and a cleavage domain. In some embodiments, the oligonucleotide is attached to a feature of the plurality of first features via the 5′ end. In some embodiments, the oligonucleotide includes from 5′ to 3′ a cleavage domain (e.g., any of the exemplary cleavage domains described herein) and a second hybridization domain. In some embodiments, the oligonucleotide includes from 5′ to 3′ a second hybridization domain and a cleavage domain (e.g., any of the exemplary cleavage domains described herein). In some embodiments, the oligonucleotide is attached to a feature of the plurality of first features via the 3′ end. In some embodiments, the oligonucleotide includes from 3′ to 5′ a cleavage domain (e.g., any of the exemplary cleavage domains described herein) and a second hybridization domain. In some embodiments, the oligonucleotide includes from 3′ to 5′ a second hybridization domain and a cleavage domain (e.g., any of the exemplary cleavage domains described herein).

In some embodiments, the second hybridization domain includes a sequence at least partially complementary to the first hybridization domain. In some embodiments, the second hybridization domain is at least at least 70% identical (e.g., at least 75% identical, at least 80% identical, at least 85% identical, at least 90% identical, at least 95% identical, or at least 99% identical) to the complementary sequence of the first hybridization domain. For example, a second hybridization domain can include a poly(T) sequence and a first hybridization sequence can include a poly(A) sequence.

In some embodiments, the second hybridization domain is at least at least 70% identical (e.g., at least 75% identical, at least 80% identical, at least 85% identical, at least 90% identical, at least 95% identical, or at least 99% identical) to the complementary sequence of the first hybridization domain. In some embodiments, a second hybridization domain is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 45 nucleotides, about 30 nucleotides to about 40 nucleotides, about 30 nucleotides to about 35 nucleotides, about 35 nucleotides to about 45 nucleotides, about 35 nucleotides to about 40 nucleotides, or about 40 nucleotides to about 45 nucleotides). In some embodiments, the length of the second hybridization domain can be used, in part, to deposit the feature on the substrate at a known location.

In some embodiments, the cleavage domain is a cleavable linker (e.g., any of the exemplary cleavable linkers described herein). In some embodiments, the cleavable linker includes a photocleavable linker, a UV-cleavable linker, a chemically cleavable linker or an enzymatic cleavable linker. In some embodiments, the cleavable linker is an enzymatic cleavable linker.

In some embodiments, a plurality of first features includes sub-pluralities of features that have different lengths of second hybridization domains on the first oligonucleotide. For example, a first sub-plurality (e.g., comprising about 50% of the total of the plurality first features) includes a second hybridization domain having a length of about 30 nucleotides and a second sub-plurality (e.g., comprising about 50% of the total of the plurality of second features) includes a second hybridization domain having a length of about 70 nucleotides. In such cases, the first sub-plurality having a second hybridization domain with a length of about 30 nucleotides can have a lower annealing temperature than the second sub-plurality having a second hybridization domain with a length of about 70 nucleotides. The difference in annealing temperature can be used to encourage hybridization of one sub-plurality over the other sub-plurality to the primers on the substrate.

In some embodiments, the method includes providing a second hybridization domain blocking moiety. In some embodiments, the second hybridization domain blocking moiety prevents the second hybridization domain from binding (e.g., coupling) to the second hybridization domain either by binding to the first hybridization domain, second hybridization domain, or both. In some embodiments, the second hybridization domain blocking moiety needs to be removed before the second hybridization domain and second hybridization domain can be coupled. Non-limiting examples of methods to remove the second hybridization domain blocking moiety from binding to the second hybridization domain, second bridging domain, or both include denaturation (e.g., increase in temperature) or enzymatic cleavage (e.g., nuclease). In some embodiments, the second hybridization domain blocking moiety is removed through passive means. For example, the binding affinity of the second hybridization domain is higher for the second hybridization domain than it is for the hybridization domain blocking moiety. In such cases, the second hybridization domain out competes the second hybridization domain blocking moiety for binding to the second hybridization domain.

In some embodiments, the second hybridization domain blocking moiety is at least partially complementary to the second hybridization domain. In some embodiments, the hybridization domain blocking moiety is at least at least 70% identical (e.g., at least 75% identical, at least 80% identical, at least 85% identical, at least 90% identical, at least 95% identical, or at least 99% identical) to the complementary sequence of the second hybridization domain. In some embodiments, binding of the second hybridization domain blocking moiety to the second hybridization domain blocks the coupling of the second hybridization domain to the second hybridization domain. In some embodiments, the method includes releasing (e.g., releasing using any of the methods described herein or know in the art) the second hybridization domain blocking moiety from the second hybridization domain.

In some embodiments, the second hybridization domain blocking moiety is at least partially complementary to the second hybridization domain. In some embodiments, the second hybridization domain blocking moiety is at least at least 70% identical (e.g., at least 75% identical, at least 80% identical, at least 85% identical, at least 90% identical, at least 95% identical, or at least 99% identical) to the complementary sequence of the second hybridization domain. In some embodiments, binding of the second hybridization domain blocking moiety to the second hybridization domain blocks the coupling of the second hybridization domain to the second hybridization domain. In some embodiments, the method includes releasing (e.g., releasing using any of the methods described herein or know in the art) the second hybridization domain blocking moiety from the second hybridization domain.

In some embodiments, the method includes a second hybridization domain blocking moiety that is at least partially complementary to the second hybridization domain and a second hybridization domain blocking moiety that is at least partially complementary to the second hybridization domain.

### (d) Bridging Probe(s)

In some embodiments, a feature of the plurality of first features includes a first bridging probe (or a plurality of bridging probes). In some embodiments, the first bridging probe is attached to a feature of the plurality of first features via the 5′ end. In some embodiments, the first bridging probe is attached to a feature of the plurality of first features via the 3′ end. In some embodiments, the first bridging probe includes a first bridging domain.

In some embodiments, the first bridging probe includes a first bridging domain and a functional sequence (e.g., any of the exemplary functional sequences described herein). In some embodiments, the functional sequence includes a sequence capable of binding to a primer used for amplification (referred to herein as the “amplification primer” or “primer used for amplification”). In some embodiments, the amplification primer is used to amplify all or part of the first bridging probe. In some embodiments, the amplification primer can be used to initiate a rolling circle amplification reaction. In some embodiments, the bridging probe to be amplified includes sequences (e.g., functional sequences, and/or bridging sequences) that enable rolling circle amplification. In some embodiments, the bridging probe is contacted with an oligonucleotide (e.g., a padlock probe). As used herein, a “padlock probe” can refer to an oligonucleotide that has, at its 5′ and 3′ ends, sequences that are complementary to adjacent or nearby target sequences on a bridging probe. Upon hybridization to the bridging probe, the two ends of the padlock probe are either brought into contact or an end is extended until the two ends are brought into contact, allowing circularization of the padlock probe by ligation (e.g., ligation using any of the methods described herein (e.g., using a T4 DNA ligase)). In some embodiments, after circularization of the oligonucleotide, rolling circle amplification can be used to amplify the bridging probe, which includes at least a bridging domain. In some embodiments, amplification of the bridging domain using a padlock oligonucleotide and rolling circle amplification increases the number of bridging domains on the substrate.

In some embodiments, the effect of the amplification of all or part of the first bridging probe is to increase the number of first bridging domains. For example, amplification of all or part of the first bridging probe using rolling circle amplification increases the number of first bridging domains. The increased number of first bridging domains in turn increases the number of sites to which the second features can couple to the first features thereby increasing the number of second features that can attach to the spatial array.

In some embodiments, the first bridging domain includes a sequence at least partially complementary to the second bridging domain. In some embodiments, the first bridging domain is at least at least 70% identical (e.g., at least 75% identical, at least 80% identical, at least 85% identical, at least 90% identical, at least 95% identical, or at least 99% identical) to the complementary sequence of the second bridging domain. In some embodiments, the first bridging domain includes a sequence that is about 5 nucleotides to about 150 nucleotides (e.g., about 5 nucleotides to about 140 nucleotides, about 5 nucleotides to about 130 nucleotides, about 5 nucleotides to about 120 nucleotides, about 5 nucleotides to about 110 nucleotides, about 5 nucleotides to about 100 nucleotides, about 5 nucleotides to about 90 nucleotides, about 5 nucleotides to about 80 nucleotides, about 5 nucleotides to about 70 nucleotides, about 5 nucleotides to about 60 nucleotides, about 5 nucleotides to about 50 nucleotides, about 5 nucleotides to about 40 nucleotides, about 5 nucleotides to about 30 nucleotides, about 5 nucleotides to about 20 nucleotides, about 5 nucleotides to about 10 nucleotides, about 10 nucleotides to about 140 nucleotides, about 10 nucleotides to about 130 nucleotides, about 10 nucleotides to about 130 nucleotides, about 10 nucleotides to about 120 nucleotides, about 10 nucleotides to about 110 nucleotides, about 10 nucleotides to about 100 nucleotides, about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 140 nucleotides, about 20 nucleotides to about 130 nucleotides, about 20 nucleotides to about 120 nucleotides, about 20 nucleotides to about 110 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 140 nucleotides, about 30 nucleotides to about 130 nucleotides, about 30 nucleotides to about 120 nucleotides, about 30 nucleotides to about 110 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 140 nucleotides, about 40 nucleotides to about 130 nucleotides, about 40 nucleotides to about 120 nucleotides, about 40 nucleotides to about 110 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 140 nucleotides, about 50 nucleotides to about 130 nucleotides, about 50 nucleotides to about 120 nucleotides, about 50 nucleotides to about 110 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 140 nucleotides, about 60 nucleotides to about 130 nucleotides, about 60 nucleotides to about 120 nucleotides, about 60 nucleotides to about 110 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 140 nucleotides, about 70 nucleotides to about 130 nucleotides, about 70 nucleotides to about 120 nucleotides, about 70 nucleotides to about 110 nucleotides, about 70 nucleotides to about 100 nucleotides, about 70 nucleotides to about 90 nucleotides, about 70 nucleotides to about 80 nucleotides, about 80 nucleotides to about 140 nucleotides, about 80 nucleotides to about 130 nucleotides, about 80 nucleotides to about 120 nucleotides, about 80 nucleotides to about 110 nucleotides, about 80 nucleotides to about 100 nucleotides, about 80 nucleotides to about 90 nucleotides, about 90 nucleotides to about 140 nucleotides, about 90 nucleotides to about 130 nucleotides, about 90 nucleotides to about 120 nucleotides, about 90 nucleotides to about 110 nucleotides, about 90 nucleotides to about 100 nucleotides, about 100 nucleotides to about 140 nucleotides, about 100 nucleotides to about 130 nucleotides, about 100 nucleotides to about 120 nucleotides, about 100 nucleotides to about 110 nucleotides, about 110 nucleotides to about 140 nucleotides, about 110 nucleotides to about 130 nucleotides, about 110 nucleotides to about 120 nucleotides, about 120 nucleotides to about 140 nucleotides, about 120 nucleotides to about 130 nucleotides, or about 130 nucleotides to about 140 nucleotides) in length.

In some embodiments, the plurality of first features includes sub-pluralities that have different lengths of first bridging domains. For example, a first sub-plurality (e.g., comprising about 50% of the total of the plurality of first features) includes a first bridging domain having a length of about 30 nucleotides and a second sub-plurality (e.g., comprising about 50% of the total of the plurality of first features) includes a first bridging domain having a length of about 70 nucleotides. In such cases, the first sub-plurality having a first bridging domain with a length of about 30 nucleotides can have a lower annealing temperature than the second sub-plurality having a first bridging domain with a length of about 70 nucleotides. The difference in annealing temperature can be used to encourage hybridization of one sub-plurality over the other sub-plurality.

In some embodiments, a feature of the plurality of second features includes a second bridging probe. In some embodiments, the second bridging probe is attached to a feature of the plurality of second features via the 5′ end. In some embodiments, the second bridging probe is attached to a feature of the plurality of second features via the 3′ end. In some embodiments, the second bridging probe includes a second bridging domain. In some embodiments, the second bridging domain includes a sequence at least partially complementary to the first bridging domain. In some embodiments, the second bridging domain is at least at least 70% identical (e.g., at least 75% identical, at least 80% identical, at least 85% identical, at least 90% identical, at least 95% identical, or at least 99% identical) to the complementary sequence of the first bridging domain. In some embodiments, the second bridging domain includes a sequence that is at about 5 nucleotides to about 150 nucleotides (e.g., about 5 nucleotides to about 140 nucleotides, about 5 nucleotides to about 130 nucleotides, about 5 nucleotides to about 120 nucleotides, about 5 nucleotides to about 110 nucleotides, about 5 nucleotides to about 100 nucleotides, about 5 nucleotides to about 90 nucleotides, about 5 nucleotides to about 80 nucleotides, about 5 nucleotides to about 70 nucleotides, about 5 nucleotides to about 60 nucleotides, about 5 nucleotides to about 50 nucleotides, about 5 nucleotides to about 40 nucleotides, about 5 nucleotides to about 30 nucleotides, about 5 nucleotides to about 20 nucleotides, about 5 nucleotides to about 10 nucleotides, about 10 nucleotides to about 140 nucleotides, about 10 nucleotides to about 130 nucleotides, about 10 nucleotides to about 130 nucleotides, about 10 nucleotides to about 120 nucleotides, about 10 nucleotides to about 110 nucleotides, about 10 nucleotides to about 100 nucleotides, about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 140 nucleotides, about 20 nucleotides to about 130 nucleotides, about 20 nucleotides to about 120 nucleotides, about 20 nucleotides to about 110 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 140 nucleotides, about 30 nucleotides to about 130 nucleotides, about 30 nucleotides to about 120 nucleotides, about 30 nucleotides to about 110 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 140 nucleotides, about 40 nucleotides to about 130 nucleotides, about 40 nucleotides to about 120 nucleotides, about 40 nucleotides to about 110 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 140 nucleotides, about 50 nucleotides to about 130 nucleotides, about 50 nucleotides to about 120 nucleotides, about 50 nucleotides to about 110 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 140 nucleotides, about 60 nucleotides to about 130 nucleotides, about 60 nucleotides to about 120 nucleotides, about 60 nucleotides to about 110 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 140 nucleotides, about 70 nucleotides to about 130 nucleotides, about 70 nucleotides to about 120 nucleotides, about 70 nucleotides to about 110 nucleotides, about 70 nucleotides to about 100 nucleotides, about 70 nucleotides to about 90 nucleotides, about 70 nucleotides to about 80 nucleotides, about 80 nucleotides to about 140 nucleotides, about 80 nucleotides to about 130 nucleotides, about 80 nucleotides to about 120 nucleotides, about 80 nucleotides to about 110 nucleotides, about 80 nucleotides to about 100 nucleotides, about 80 nucleotides to about 90 nucleotides, about 90 nucleotides to about 140 nucleotides, about 90 nucleotides to about 130 nucleotides, about 90 nucleotides to about 120 nucleotides, about 90 nucleotides to about 110 nucleotides, about 90 nucleotides to about 100 nucleotides, about 100 nucleotides to about 140 nucleotides, about 100 nucleotides to about 130 nucleotides, about 100 nucleotides to about 120 nucleotides, about 100 nucleotides to about 110 nucleotides, about 110 nucleotides to about 140 nucleotides, about 110 nucleotides to about 130 nucleotides, about 110 nucleotides to about 120 nucleotides, about 120 nucleotides to about 140 nucleotides, about 120 nucleotides to about 130 nucleotides, or about 130 to about 140 nucleotides) in length.

In some embodiments, the plurality of second features includes sub-pluralities that have different lengths of second bridging domains. For example, a first sub-plurality (e.g., comprising about 50% of the total of the plurality of second features) includes a second bridging domain having a length of about 30 nucleotides and a second sub-plurality (e.g., comprising about 50% of the total of the plurality of second features) includes a second bridging domain having a length of about 70 nucleotides. In such cases, the first sub-plurality having a second bridging domain with a length of about 30 nucleotides can have a lower annealing temperature than the second sub-plurality having a second bridging domain with a length of about 70 nucleotides. The difference in annealing temperature can be used to encourage hybridization of one sub-plurality over the other sub-plurality.

In some embodiments, the first bridging domain includes a sequence that is a different length compared to other bridging domains. In some embodiments, the second bridging domain includes a sequence that is a different length compared to other bridging domains. In some embodiments, the first bridging domain and the second bridging domain are the same length. In some embodiments, the first bridging domain and the second bridging domain are both about 10 nucleotides to about 30 nucleotides in length. In some embodiments, the first bridging domain and the second bridging domain are both about 30 nucleotides to about 50 nucleotides in length. In some embodiments, the first bridging domain and the second bridging domain are both about 50 nucleotides to about 70 nucleotides in length. In some embodiments, the first bridging domain and the second bridging domain are both about 70 nucleotides to about 90 nucleotides in length. In some embodiments, the first bridging domain and the second bridging domain are both at least 90 nucleotides in length.

In some embodiments, the method of producing the spatial array includes temperature modulation to encourage or discourage coupling of the first bridging domain to the second bridging domain (e.g., temperature modulation based on nucleotide sequence length). In some embodiments, annealing temperature is used to modulate the coupling of the first bridging domain to the second bridging domain. In some embodiments, the difference in annealing temperature can be used to encourage hybridization between a first bridging domain and a second bridging domain that have similar annealing temperatures. In some embodiments, a first bridging domain and a second bridging domain each have an annealing temperature that is about 35° C. to about 45° C., about 36° C. to about 44° C., about 37° C. to about 43° C., about 38° C. to about 42° C., or about 39° C. to about 41° C. In some embodiments, a first bridging domain and a second bridging domain each have an annealing temperature that is about 45° C. to about 55° C., about 46° C. to about 54° C., about 47° C. to about 53° C., about 48° C. to about 52° C., or about 49° C. to about 51° C. In some embodiments, a first bridging domain and a second bridging domain each have an annealing temperature that is about 55° C. to about 65° C., about 56° C. to about 64° C., about 57° C. to about 63° C., about 58° C. to about 62° C., or about 59° C. to about 61° C.

In some embodiments, the method includes providing a bridging domain blocking moiety. In some embodiments, the bridging domain blocking moiety prevents the first bridging domain from binding (e.g., coupling) to the second bridging domain either by binding to the first bridging domain, second bridging domain, or both. In some embodiments, the bridging domain blocking moiety needs to be removed before the first bridging domain and second bridging domain can be coupled. Non-limiting examples of methods to remove the bridging domain blocking moiety from binding to the first bridging domain, second bridging domain, or both include denaturation (e.g., increase in temperature) or enzymatic cleavage (e.g., nuclease). In some embodiments, the bridging domain blocking moiety is removed through passive means. For example, the binding affinity of the first bridging domain is higher for the second bridging domain than it is for the bridging domain blocking moiety. In such cases, the second bridging domain out competes the bridging domain blocking moiety for binding to the first bridging domain.

In some embodiments, the bridging domain blocking moiety is at least partially complementary to the first bridging domain. In some embodiments, the bridging domain blocking moiety is at least at least 70% identical (e.g., at least 75% identical, at least 80% identical, at least 85% identical, at least 90% identical, at least 95% identical, or at least 99% identical) to the complementary sequence of the first bridging domain. In some embodiments, binding of the bridging domain blocking moiety to the first bridging domain blocks the coupling of the first bridging domain to the second bridging domain. In some embodiments, the method includes releasing (e.g., releasing using any of the methods described herein or know in the art) the bridging domain blocking moiety from the first bridging domain.

In some embodiments, the bridging domain blocking moiety is at least partially complementary to the second bridging domain. In some embodiments, the bridging domain blocking moiety is at least at least 70% identical (e.g., at least 75% identical, at least 80% identical, at least 85% identical, at least 90% identical, at least 95% identical, or at least 99% identical) to the complementary sequence of the second bridging domain. In some embodiments, binding of the bridging domain blocking moiety to the second bridging domain blocks the coupling of the second bridging domain to the first bridging domain. In some embodiments, the method includes releasing (e.g., releasing using any of the methods described herein or know in the art) the bridging domain blocking moiety from the second bridging domain.

In some embodiments, the method includes a first bridging domain blocking moiety that is at least partially complementary to the first bridging domain and a second bridging domain blocking moiety that is at least partially complementary to the second bridging domain.

### (e) First Capture Probe(s) and Second Capture Probe(s)

In some embodiments, the spatial array includes a feature of the plurality of first features that includes a first capture probe (or a plurality of first capture probes) including a first spatial barcode and a first capture domain and a second feature of the plurality of second features that includes a second capture probe (or a plurality of second capture probes) including a second spatial barcode and a second capture domain. In some embodiments, the first capture probe includes one or more of a capture domain, a cleavage domain, a spatial barcode, a unique molecular identifier, or any other aspect of a capture probe as disclosed herein, or any combination thereof. In some embodiments, the second capture probe includes one or more of a capture domain, a cleavage domain, a spatial barcode, a unique molecular identifier, or any other aspect of a capture oligonucleotide probe as disclosed herein, or any combination thereof. In some embodiments, the first spatial barcode and the second spatial barcode are identical. In some embodiments, the first spatial barcode and the second spatial barcode are different. In some embodiments, the first capture domain and the second capture domain are the same. For example, in some embodiments, the first capture domain and the second capture domain each include a poly(T) domain. In some embodiments, the first capture domain and the second capture domain are different.

### (f) First Feature(s) and Second Feature(s)

Provided herein are methods of preparing a spatial array that includes hybridizing a feature to a primer attached to a substrate. As used herein, a “feature” includes an entity that acts as a support or repository for at least an oligonucleotide, a capture probe, and/or a bridging probe. In some embodiments, functionalized features include one or more capture probe(s). Examples of features include, but are not limited to, a bead, a spot of any two- or three-dimensional geometry (e.g., an ink jet spot, a masked spot, a square on a grid), a well, and a hydrogel pad. In some embodiments, a feature is deposited on the substrate at a known location. In some embodiments, a feature is deposited on the array using printing or spotting. Jet printing of biopolymers is described, for example, in PCT Patent Application Publication No. WO 2014/085725. Jet printing of polymers is described, for example, in de Gans et al., Adv Mater. 16(3): 203-213 (2004).

In some embodiments, a feature of the plurality of first features includes an oligonucleotide, a first capture probe, and a first bridging probe. In some embodiments, a first feature refers to a feature of a plurality of first features. In some embodiments, a first feature includes an additional first capture probe. In some embodiments, the first capture probe and the additional first capture probe each have the same spatial barcode sequence. In some embodiments, the additional first capture probe includes a different capture domain from the first capture probe. In some embodiments, a first feature includes a third capture probe, a fourth capture probe, a fifth capture probe, a sixth capture probe, a seventh capture probe, an eighth capture probe, a ninth capture or ten or more capture probes. In such cases, each of the capture probes include the same spatial barcode. In some embodiments where a feature includes a first capture probe and an additional first capture probe that each include a different capture domain, each of the first and second capture probes are used to capture a different analyte. For example, a first capture probe includes a poly(T) capture domain that can be used to bind to a poly(A) signal on an mRNA molecule and a second capture probe includes a homopolymeric sequence present in a genomic DNA molecule.

In some embodiments, a feature of the plurality of first features includes a known combination of first capture probe, a first oligonucleotide, and first bridging probe, wherein determining the location of the first feature is based on the known combination.

In some embodiments, a feature of the plurality of first features is a first bead. As used herein, a first “bead” or a second “bead,” or additional “beads” can be a particle. A bead can be porous, non-porous, solid, semi-solid, and/or a combination thereof. In some embodiments, a bead can be dissolvable, disruptable, and/or degradable, whereas in certain embodiments, a bead is not degradable. In some embodiments, the first bead has a diameter of about 0.1 μm to about 5 μm, 0.5 μm to about 4 μm, about 1 μm to about 10 μm, about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 90 μm to about 100 μm, about 80 μm to about 100 μm, about 70 μm to about 100 μm, about 60 μm to about 100 μm, about 50 μm to about 100 μm, about 40 μm to about 100 μm, about 30 μm to about 100 μm, about 20 μm to about 100 μm, or about 10 μm to about 100 μm. In some embodiments, a spatial array comprising a plurality of features comprises first and second beads, where the first and second beads are of the same or different average diameters. In some embodiments, the spatial array may further comprise third and optionally, fourth beads, where the third and fourth beads are of the same or different average diameters. In some embodiments, the spatial array may further comprise third and optionally, fourth beads, where the third and fourth beads are of the same or different average diameters as compared to the first and second beads.

In some embodiments, a feature (e.g., a bead) of the plurality of first features is provided to the spatial array in a manner where the feature has a known location on the substrate. For example, a feature of the plurality of first features is deposited on the substrate using printing or spotting. In some embodiments, a feature (e.g., a bead) of the plurality of first features is provided to the substrate in a manner where the coupling of the first hybridization domain to the second hybridization domain determines the location of the feature (e.g., the bead) on the spatial array. In some embodiments, a feature of the plurality of first features is provided to the substrate in a particular x- and/or y-coordinate pattern wherein the feature is deposited on the substrate at a known location.

In some embodiments, a feature of the plurality of second features includes an oligonucleotide, a second capture probe, and a second bridging probe. In some embodiments, a second feature refers to a feature of a plurality of second features. In some embodiments, a second feature includes an additional second capture probe. In some embodiments, the second capture probe and the additional second capture probe each have the same spatial barcode sequence. In some embodiments, the additional second capture probe includes a different capture domain from the second capture probe. In some embodiments, a second feature includes a third capture probe, a fourth capture probe, a fifth capture probe, a sixth capture probe, a seventh capture probe, an eighth capture probe, a ninth capture or ten or more capture probes. In such cases, each of the capture probes include the same spatial barcode. In some embodiments where a feature includes a second capture probe and an additional second capture probe that each include a different capture domain, each of the second and additional second capture probes are used to capture a different analyte. For example, a second capture probe includes a poly(T) capture domain that can be used to bind to a poly(A) signal on an mRNA molecule and an additional second capture probe includes a homopolymeric sequence present in a genomic DNA molecule.

In some embodiments, a feature of the plurality of second features includes a known combination of second capture probe and second bridging probe, wherein determining the location of the second feature is based on the known combination.

In some embodiments, a feature of the plurality of second features is a second bead. In some embodiments, the second bead has a diameter of about 0.1 μm to about 5 μm, 0.5 μm to about 4 μm, about 1 μm to about 10 μm, about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 90 μm to about 100 μm, about 80 μm to about 100 μm, about 70 μm to about 100 μm, about 60 μm to about 100 μm, about 50 μm to about 100 μm, about 40 μm to about 100 μm, about 30 μm to about 100 μm, about 20 μm to about 100 μm, or about 10 μm to about 100 μm.

In some embodiments, a feature (e.g., a bead) of the plurality of second features is provided to the spatial array in a manner where the feature has a known location on the substrate. For example, a feature of the plurality of second features is deposited on the substrate using printing or spotting. In some embodiments, a feature (e.g., a bead) of the plurality of second features is provided to the substrate in a manner where the coupling of the second bridging domain to the second bridging domain determines the location of the feature (e.g., the bead) on the spatial array. In some embodiments, a feature of the plurality of second features is provided to the substrate in a particular x- and/or y-coordinate pattern wherein the feature is deposited on the substrate at a known location.

### (g) Spatial Analysis Using High Resolution Spatial Arrays

In some embodiments, a method for spatial analysis of a biological analyte in a biological sample includes using the spatial array prepared according to the methods described herein. In some embodiments, a method for spatial analysis using the spatial array prepared according to the methods described herein includes capturing an analyte of a biological sample with a first capture probe of the plurality of first features and/or a second capture probe of the plurality of second features; and determining a location of the captured analyte in the biological sample based on the location of the first and/or second feature in the spatial array. In some embodiments, the method includes contacting the spatial array with the biological sample and allowing the analyte to interact with the first and/or second capture probes. In some embodiments, the determining step includes amplifying all or part of the analyte specifically bound to the capture domain of the first and/or second capture probes. In some embodiments, the method includes amplifying all or part of the analyte using isothermal amplification. In some embodiments, the method includes amplifying all or part of the analyte using non-isothermal amplification. In some embodiments, the amplifying creates an amplifying product that includes (i) all or part of sequence of the analyte specifically bound to the first capture domain and/or the second capture domain, or a complement thereof, and (ii) all or a part of the sequence of the first spatial barcode and/or the second spatial barcode, or a complement thereof. In some embodiments, the associating step also includes determining (i) all or part of the sequence of the first spatial barcode and (ii) all or part of the sequence of the second spatial barcode and using the determined sequence of (i) and (ii) to identify the location of first feature and the location of the second feature in the spatial array. In some embodiments, the determining step includes sequencing. A non-limiting example of sequencing that can be used to determine the sequence of the analyte and/or spatial barcodes (e.g., first and/or second spatial barcode) is in situ sequencing. In some embodiments, in situ sequencing is performed via sequencing-by-synthesis (SBS), sequential fluorescence hybridization, sequencing by ligation, nucleic acid hybridization, or high-throughput digital sequencing techniques. In some embodiments the analyte is RNA or DNA. In some embodiments, the analyte is protein.

More particularly, after an analyte (e.g., a first analyte, a second analyte, etc.) has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to a capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, the methods provided herein include spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by uracil-DNA glycosylase, apurinic/apyrimidinic (AP) endonuclease (APE1), uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., a reverse transcription step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the proximal capture probes. In some embodiments, the capture probe is extended using one or more DNA polymerases.

In some embodiments, a capture domain of a capture probe includes a nucleic acid sequence for producing a complementary strand of a nucleic acid hybridized to the capture probe, e.g., a primer for DNA polymerase and/or reverse transcription. The nucleic acid (e.g., DNA and/or cDNA) molecules generated by the extension reaction incorporate the sequence of the capture probe. Extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9°N) DNA ligase (9°N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using an amplification primer including the affinity group. In some embodiments, the amplification primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the capture probe can be amplified or copied, creating a plurality of cDNA molecules. In some embodiments, cDNA can be denatured from the capture probe template and transferred (e.g., to a clean tube or microwell plate) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (e.g., Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. A skilled artisan will understand that additional or alternative sequences used by other sequencing instruments or technologies are also equally applicable for use in the aforementioned methods as the current methods are not limited to any a particular sequencing platform.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze the barcoded analyte or moiety. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (h) Kits

In some embodiments, also provided herein are kits that include one or more reagents to prepare a spatial array as described herein. In some instances, the kit includes a substrate including a plurality of primers including a hybridization domain. In some instances, the kit further comprises a plurality of first features and a plurality of second features.

A non-limiting example of a kit used to perform any of the methods described herein includes: (a) an array including a plurality of primers; (b) a plurality of first features including an oligonucleotide, a first capture probe, and a first bridging probe; (c) a plurality of second features including a second capture probe, and a second bridging probe; and (d) instructions for performing any of the methods described herein. In some embodiments, the kits can include one or more enzymes for performing any of the methods described herein, including but not limited to, a DNA polymerase, a reverse transcriptase, a ligase, an endonuclease, a protease, or a combination thereof.

In some embodiments, also provided herein are kits that include one or more reagents to detect one or more analytes in a biological sample. In some embodiments, the kit includes an array including a plurality of primers hybridized to a plurality of first features, wherein the first features are hybridized to a plurality of second features. Another non-limiting example of a kit used to perform any of the methods described herein includes: (a) an array including a plurality of primers hybridized to a plurality of first features, wherein the first features are hybridized to a plurality of second features, wherein a feature of the first plurality of features includes an oligonucleotide, a first capture probe, and a first bridging probe, wherein a feature of the second plurality of features includes a second capture probe and a second bridging probe; and (b) instructions for performing any of the methods described herein.

### (i) Compositions

In some instances, disclosed herein are compositions that are used to carry out the methods described herein. In another aspect, this disclosure includes compositions including a substrate that includes (a) a plurality of primers attached to a surface of the substrate, wherein a primer of the plurality of primers includes a first hybridization domain; and (b) a plurality of first features, wherein a feature of the plurality of first features includes an oligonucleotide, a first capture probe, and a first bridging probe, wherein: (i) the oligonucleotide includes a second hybridization domain, wherein the second hybridization domain is capable of hybridizing to the first hybridization domain; (ii) the first capture probe includes a first spatial barcode and a first capture domain, wherein the first capture domain is capable of binding to a first analyte from a biological sample; and (iii) the first bridging probe includes a first bridging domain, wherein the first bridging domain is capable of binding to a second bridging domain, wherein a feature of the first plurality of features is coupled to a primer of the plurality of primers via hybridization of the first hybridization domain to the second hybridization domain.

In another aspect, this disclosure includes compositions that includes (a) a plurality of primers attached to a surface of the substrate, wherein a primer of the plurality of primers includes a first hybridization domain; (b) a plurality of first features, wherein a feature of the plurality of first features includes an oligonucleotide, a first capture probe, and a first bridging probe, wherein: (i) the oligonucleotide includes a second hybridization domain, wherein the second hybridization domain is capable of hybridizing to the first hybridization domain; (ii) the first capture probe includes a first spatial barcode and a first capture domain, wherein the first capture domain is capable of binding to a first analyte from a biological sample; and (iii) the first bridging probe includes a first bridging domain, wherein the first bridging domain is capable of binding to a second bridging domain; and (c) a plurality of second features, wherein a feature of the plurality of second features includes a second capture probe and a second bridging probe, wherein: (i) the second capture probe includes a second spatial barcode and a second capture domain, wherein the second capture domain is capable of binding to a second analyte from the biological sample; and (ii) the second bridging probe includes a second bridging domain, wherein the second bridging domain is capable of binding to the first bridging domain, wherein a feature of the first plurality of features is coupled to a primer of the plurality of primers via hybridization of the first hybridization domain to the second hybridization domain, wherein a feature of the second plurality of features is coupled to a feature of the first plurality of features via hybridization of the second bridging domain to the first bridging domain.

In some embodiments, the compositions also include an analyte bound to the first and/or second capture probes. In some embodiments, the composition also includes an analyte bound to the first and/or second capture probes, where the capture probe has been extended using the captured analyte as a template (e.g., as a template in a nucleic acid extension reaction.

### EXAMPLES

### Example 1—Preparing a Spatial Array

This example provides an exemplary method for preparing a spatial array. In a non-limiting example, a plurality of primers on a substrate can be used to guide features that include capture probes onto the substrate. A second set of features that can hybridize to the first features and that also include capture probes are then added to the substrate. The second set of features increase the resolution of the array as they are deposited on the substrate in locations or spaces between the primers and/or the first features.

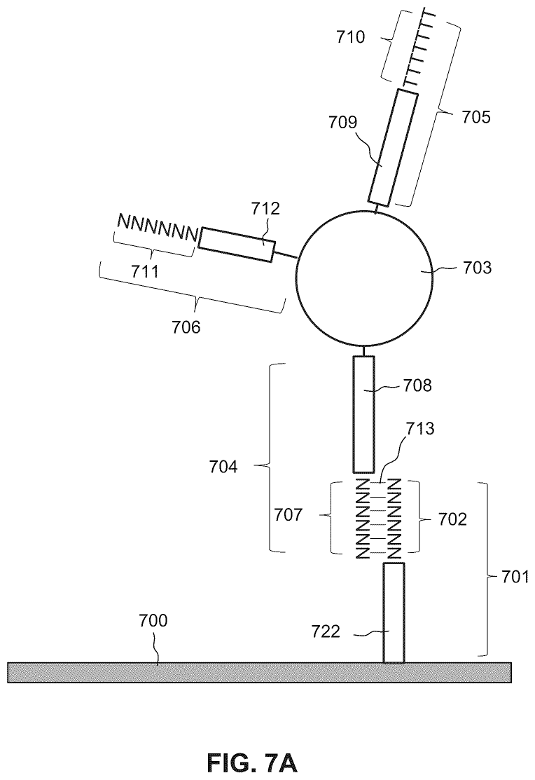

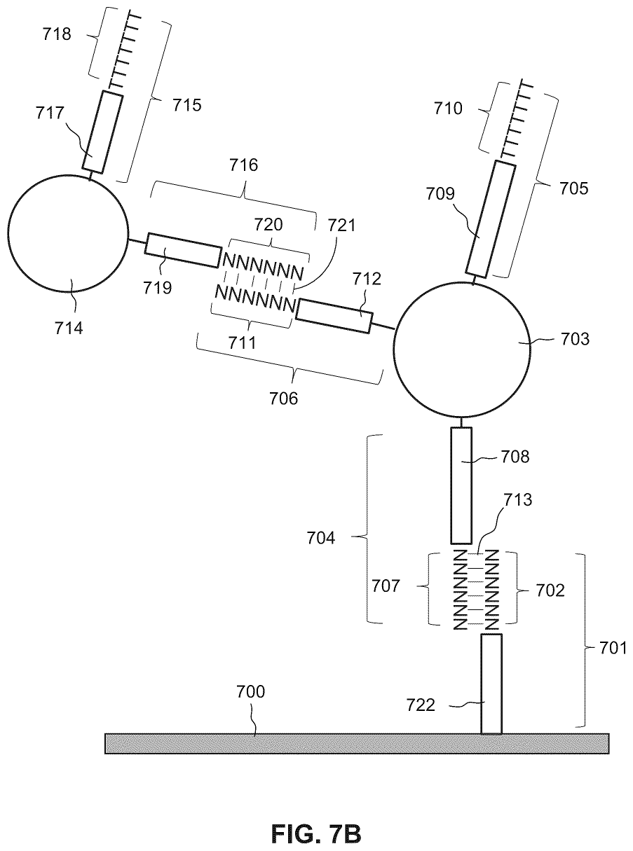

As seen in FIG. 7A, a substrate 700 includes a primer 701 affixed to the surface of the substrate. The primer 701 includes a first hybridization domain 702. The primer with a known first hybridization domain 702 and a functional domain 722 is deposited on the array in a known location using an inkjet printer. Next, a plurality of first features are provided. A feature 703 of the plurality of first features includes an oligonucleotide 704, a first capture probe 705, and a first bridging probe 706. The oligonucleotide 704 includes a second hybridization domain 707 that is capable of hybridizing to the first hybridization domain and a cleavage domain 708. The first capture probe 705 includes a first spatial barcode 709 and a first capture domain 710, where the first capture domain is capable of binding to an analyte. The first bridging probe 706 includes a first bridging domain 711 that is capable of binding to a second bridging domain, and a functional domain 712. The feature 703 of the plurality of first features is attached to the primer 701 on the substrate by hybridizing (as indicated by numeral 713) the second hybridization domain 707 to the first hybridization domain 702. The location of the feature 703 from the plurality of first features in the spatial array is determined based on the location of the first hybridization domain 702 of the primer 701 to which the first feature hybridizes.

Next, as seen in FIG. 7B, a plurality of second features is provided. A feature 714 of the plurality of second features includes a second capture probe 715 and a second bridging probe 716. The second capture probe 715 includes a second spatial barcode 717 and a second capture domain 718, where the second capture domain 718 is capable of binding to an analyte. The second bridging probe 716 includes a functional domain 719 and a second bridging domain 720, where the second bridging domain 720 is capable of binding to the first bridging domain 711. The feature 714 of the plurality of second features is attached to the feature 703 of the plurality of the first features by hybridizing (as indicated by numeral 721) the second bridging probe 716 to the first bridging probe 706. The location of the feature 714 of the plurality of second features in the spatial array is determined based on the location of the first spatial barcode and the second spatial barcode in the array. Additionally, the second set of features can hybridize to other features from the plurality of second features via hybridization of the second bridging domain to second bridging domains located on other second features, thereby generating a high resolution array by “filling” the spaces between the printed primers on the spatial array. In such cases, the second set of features can include an additional bridging probe that includes a bridging domain capable of hybridizing specifically to other additional bridging probes located on other second features.

### Example 2—Spatial Profiling with a High Resolution Array

This example provides an exemplary method for spatial analysis of a biological analyte in a biological sample using a high resolution spatial array (e.g., an array having a resolution beyond the limits of inkjet print technology) prepared according to the methods described herein. In a non-limiting example, a high resolution spatial array is provided for spatial analysis where the spatial array is constructed by providing a second set of features to a spatial array to “fill” the spaces between the printed primers on the spatial array. A spatial array is prepared with a substrate having printed primer features of 30 microns, and the second features allow for increased resolution of the features to 20 microns, or smaller.

As seen in FIG. 7A and FIG. 7B, the plurality of the first features are coupled to the array via hybridization between an oligonucleotide on a feature and a primer that is affixed to the substrate. The plurality of second features are provided to the spatial array and hybridize to the features of the plurality of first features via a first bridging probe on the first feature and a second bridging probe on the second feature. The hybridizing of the second set of features to the first set of features has the effect of increasing the resolution of the array by “filling” in the spaces between the first features and/or the printed primers on the spatial array.

The high resolution spatial array generated in FIG. 7A and FIG. 7B is contacted with a biological sample under conditions where a biological analyte from the biological sample interacts with the capture probes on the plurality of first features and/or the plurality of second features on the spatial array. The location of the analyte in the biological sample is resolved by determining (i) all or a part of the sequence of the analyte specifically bound to the first capture domain and/or the second capture domain, or a complement thereof, and (ii) all or a part of the sequence of the first spatial barcode and/or the second spatial barcode, or a complement thereof, and using the determined sequence of (i) and (ii) to identify the location of the analyte in the biological sample.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining a location of a first analyte in a biological sample, the method comprising:
(a) contacting a substrate comprising a spatial array with the biological sample, wherein the spatial array comprises:
(i) a plurality of primers attached to a surface of the substrate, wherein a primer of the plurality of primers comprises a first hybridization domain; and
(ii) a plurality of first features, wherein a first feature of the plurality of first features comprises an oligonucleotide, a first capture probe, and a first bridging probe, wherein:
the oligonucleotide comprises a second hybridization domain, wherein the second hybridization domain is hybridized to the first hybridization domain;
the first capture probe comprises a first spatial barcode and a first capture domain, wherein the first capture domain is capable of hybridizing to the first analyte from the biological sample; and
the first bridging probe comprises a first bridging domain, wherein the first bridging domain is capable of hybridizing to a second bridging domain;
(b) hybridizing the first analyte to the first capture probe; and
(c) determining (i) all or a part of a sequence of the first analyte, or a complement thereof, and (ii) a sequence of the first spatial barcode, or a complement thereof, and using the determined sequence of (i) and (ii) to determine the location of the first analyte in the biological sample.

2. The method of claim 1, wherein the method further comprises extending the first capture probe using the first analyte as a template to generate a first extended capture probe.

3. The method of claim 1, wherein step (c) comprises amplifying all or part of the first analyte hybridized to the first capture domain, wherein the amplifying creates an amplification product comprising (i) all or part of the first analyte hybridized to the first capture domain, or a complement thereof, and (ii) the sequence of the first spatial barcode, or a complement thereof.

4. The method of claim 1, wherein step (c) comprises sequencing.

5. The method of claim 1, wherein the first capture probe further comprises one or more first functional domains, a first unique molecular identifier, a first cleavage domain, or a combination thereof.

6. The method of claim 1, wherein the first bridging domain is about 10 nucleotides to about 90 nucleotides in length.

7. The method of claim 1, wherein the spatial array further comprises a plurality of second features, wherein a second feature of the plurality of second features comprises a second capture probe and a second bridging probe, wherein:
the second capture probe comprises a second spatial barcode and a second capture domain, wherein the second capture domain is capable of hybridizing to a second analyte from the biological sample; and
the second bridging probe comprises the second bridging domain hybridized to the first bridging domain.

8. The method of claim 7, further comprising determining a location of the second analyte in the biological sample, the method comprising:
(d) hybridizing the second analyte to the second capture probe; and
(e) determining (iii) all or a part of a sequence of the second analyte, or a complement thereof, and (iv) a sequence of the second spatial barcode, or a complement thereof, and using the determined sequence of (iii) and (iv) to determine the location of the second analyte in the biological sample.

9. The method of claim 7, wherein step (e) comprises amplifying all or part of the second analyte hybridized to the second capture domain, wherein the amplifying creates an amplification product comprising (iii) all or part of the second analyte hybridized to the second capture domain, or a complement thereof, and (iv) the sequence of the second spatial barcode, or a complement thereof.

10. The method of claim 7, wherein step (e) comprises sequencing (iii) all or part of the second analyte hybridized to the second capture domain, or a complement thereof, and (iv) the sequence of the second spatial barcode, or a complement thereof.

11. The method of claim 7, wherein the second capture probe further comprises one or more second functional domains, a second unique molecular identifier, a second cleavage domain, or a combination thereof.

12. The method of claim 7, wherein the second bridging domain is about 10 nucleotides to about 90 nucleotides in length.

13. The method of claim 7, wherein the method further comprises extending the second capture probe using the second analyte as a template to generate a second extended capture probe.

14. The method of claim 1, further comprising delivering one or more permeabilization reagents selected from proteinase K, pepsin, or collagenase.

15. The method of claim 1, further comprising imaging the biological sample.

16. The method of claim 1, wherein the biological sample was previously stained using immunofluorescence, immunohistochemistry, hematoxylin, and/or eosin.

17. The method of claim 1, wherein the biological sample is a formalin-fixed, paraffin-embedded (FFPE) tissue sample, a frozen tissue sample, or a fresh tissue sample.

18. The method of claim 1, wherein the biological sample is a tissue section.

19. The method of claim 1, wherein the first analyte is RNA or DNA.

20. The method of claim 7, wherein the second analyte is RNA or DNA.
